(19) United States
(12) Patent Application Publication
(10) Pub. No.: US 2005/0106561 A1
(43) Pub. Date: May 19, 2005

(54) Method for predicting the severity of viral hepatitis type B in patients treated with an anti-viral agent, and a kit for diagnosing viral hepatitis type B

(76) Inventors: Yoshiyuki Suzuki, Tokyo (JP); Hiromitsu Kumada, Tokyo (JP)

Correspondence Address: CHOATE, HALL & STEWART LLP, EXCHANGE PLACE, 53 STATE STREET, BOSTON, MA, 02109, US

(21) Appl. No.: 10/913,910

(22) Filed: Aug. 6, 2004

(30) Foreign Application Priority Data
Nov. 13, 2003 (JP) 2003-383408

(51) Int. Cl. C12Q 1/70

(52) U.S. Cl. 435/5

(57) ABSTRACT

Severity of viral hepatitis type B in patients treated with an anti-viral agent including lamivudine is predicted on the basis of the quantified value of cytochrome c in body fluid of the patients. Cytochrome c is preferably quantified immunochemically.

### BACKGROUND OF THE INVENTION

The present invention relates to a method for predicting the severity of viral hepatitis type B in patients treated with an anti-viral agent including lamivudine by quantifying cytochrome c, and a diagnostic kit used in the method.

Lamivudine has been approved as a remedy for viral hepatitis type B and has provided good results in the treatment of viral hepatitis type B that had been cureless in the past (Lai C L, Chien R N, Leung N W Y, et al: A one-year trial of Lamivudine for chronic hepatitis B. Asia Hepatitis Lamivudine Study Group. N Engl J Med 339 : 61-68, 1998, Dienstag J L, Schiff E R, Wright T L, et al: Lamivudine as initial treatment for chronic hapatitis B in the United States. N Eng J Med 341 : 1256-1263, 1999, Suzuki Y, Kumada H, Ikeda K, et al: Histological changes in liver biopsies after one year of lamivudine treatment in patients with chronic hepatitis B infection. J Hepatol 30 : 743-748, 1999, Tsubota A, Arase Y, Saitoh S, et al: Lamivudine therapy for spontaneously occurring severe acute exacerbation in chronic hapatitis B virus infection: a preliminary study. Am J Gastroenterol 96 : 557-562, 2001).

However, there are occasionally found cases where hepatitis becomes fulminant after quitting administration of lamivudine (Honkoop P, de Man R A, Heijtink R A, et al: Hepatitis B reactivation after lamivudine. Lancet 346(8983) : 1156-1157, 1995) and cases where lamivudine-resistant virus appears and hepatitis becomes fulminant during lamivudine treatment (Hiroshi Iwanaga et al., Acta Hepatologica Japonica 41:660-664, 2000), and the difficulty in determining the timing of quitting the administration prevents prevalence of the treatment with lamivudine. In any case, immunosuppressive therapy with steroid including prednisolone, anti-viral therapy with interferon, or combined therapy with nucleoside analogues including adefovir and entecavir is effective if progression to fulminant hepatitis occurs (Koji Adachi et al., Acta Hepatologica Japonica 43:322-326, 2002; Fumitaka Suzuki et al., Acta Hepatologica Japonica 43:432-435, 2002; and Kazuaki Inoue et al., Acta Hepatologica Japonica 44:428-428, 2003). However, it is difficult to predict progression to fulminant hepatitis by the amount of HB antigen or biochemical markers of hepatic function, which have been conventionally measured. Hence, a diagnostic reagent for predicting progression to fulminant hepatitis has long been desired.

### SUMMARY OF THE INVENTION

An object of the present invention is to develop a method for predicting the severity of viral hepatitis type B in patients treated with an anti-viral agent including lamivudine, and a diagnostic kit used in the method.

The inventors of the present invention considered that apoptosis of hepatocytes in viral hepatitis type B could be measured by cytochrome c in blood according to a similar manner as in WO 01/35093, in which apoptosis in a living body occurring in hemophagocytic syndrome (HPS), graft versus host disease (GVHD), acute lymphoid leukemia and influenza encephalitis had been measured. Then, they have established an ELISA method for quantifying cytochrome c, and found that a patient with a large amount of cytochrome c in blood has a higher severity of viral hepatitis type B. Thus, they have accomplished the present invention.

That is, the present invention relates to the followings.
(1) A method for predicting severity of viral hepatitis type B in patients treated with an anti-viral agent, wherein cytochrome c in body fluid of the viral hepatitis type B patients is quantified.
(2) A method according to (1), wherein the anti-viral agent is lamivudine.
(3) A method according to (1) or (2) comprising the steps of:
(a) quantifying cytochrome c in body fluid of the viral hepatitis type B patients treated with an anti-viral agent; and
(b) determining that the severity is high when the quantified value of cytochrome c is high.
(4) A method according to (1) or (2), wherein cytochrome c is quantified by an immunochemical method.
(5) A diagnostic kit for predicting severity of viral hepatitis type B in patients treated with an anti-viral agent, comprising a reagent for quantifying cytochrome c.
(6) A kit according to (5), wherein the anti-viral agent is lamivudine.

According to the present invention, it has become possible to predict the severity of viral hepatitis type B in patients treated with an anti-viral agent including lamivudine by quantifying cytochrome c in body fluid.

### DETAILED DESCRIPTION OF THE INVENTION

Hereafter, embodiments of the present invention will be explained in detail.

In the present specification, the term “body fluid” refers to blood, plasma, serum, cerebrospinal fluid or the like collected from a living body.

The methods for quantifying cytochrome c include an immunochemical method, electrophoresis and chromatography. Electrophoresis includes: a method in which polyacrylamide electrophoresis is performed to detect cytochrome c as a band; and a method in which capillary electrophoresis is performed to detect cytochrome c as a peak. Further, chromatography includes a method in which high performance liquid chromatography is performed to detect cytochrome c as a peak. If necessary, cytochrome c may be labeled with fluorescence in order to increase sensitivity. However, the present invention is not limited to these examples.

As the method for quantifying cytochrome c, an immunochemical method is preferable in view of sensitivity and simplicity. “Immunochemical methods” include a method of quantifying cytochrome c by using an antibody against cytochrome c. The immunochemical methods include various methods such as a competitive method in which cytochrome c is labeled, a sandwich method in which an antibody is labeled, and a latex bead method in which agglutination of antibody-coated beads is observed. Any method is included in the methods of the present invention so long as an antibody against cytochrome c is used. The antibody may be a monoclonal or polyclonal antibody. The labeling methods include various methods such as labeling with a radioactive isotope, labeling with a compound exhibiting electro-chemiluminescence, labeling with fluorescence, labeling with an enzyme and labeling with biotin. However, the present invention is not limited to these examples. The methods for preparing and labeling an antibody are described in, for example, “Zoku Seikagaku Jikken Kouza 5: Meneki Seikagaku Kenkyuhou (Supplemental Biochemical Experiment Lectures 5: Immunobiochemical research methods)” (edited by The Japanese Biochemical Society and issued by Tokyo Kagaku Dozin Co., Ltd.) or “Shin Seikagaku Jikken Kouza 12: Bunshi Menekigaku III (New Biochemical Experiment Lectures 12: Molecular Immunology III)” (edited by The Japanese Biochemical Society and issued by Tokyo Kagaku Dozin Co., Ltd.).

A sandwich method as an example of the immunochemical method for quantifying cytochrome c will be explained below step by step.
1) An antibody against cytochrome c is immobilized on beads or a cup. The antibody to be immobilized may be either a polyclonal or monoclonal antibody so long as the antibody is an antibody which provides required sensitivity, that is, sensitivity that enables quantification of preferably 0.5 ng/mL, more preferably 0.1 ng/mL of cytochrome c. A commercially available antibody, for example, clone: 6H2.B4, (Becton, Dickinson and Company) or clone: 2B5F8 (TECHNE Inc.) may be used as a monoclonal antibody. The beads may be microbeads, and magnetic microbeads are preferable. The immobilization may be achieved by a covalent or non-covalent bond. Usually, a blocking operation using a protein such as bovine serum albumin (BSA) or casein, or surfactant such as Tween 20 is performed in order to block nonspecific binding sites on the beads or cup.
2) A sample is diluted with buffer containing a protein such as BSA or casein or surfactant such as Tween 20 if required, and added to the beads or the cup. Further, a known amount of cytochrome c is diluted and added in the same way.
3) The beads or the cup is preferably washed with buffer containing surfactant such as Tween 20, and then a labeled anti-cytochrome c antibody diluted with buffer containing a protein such as BSA or casein or surfactant such as Tween 20 is preferably added thereto. The labeled antibody maybe either a polyclonal or monoclonal antibody so long as the antibody is an antibody which provides required sensitivity, that is, sensitivity that enables quantification of preferably 0.5 ng/mL, more preferably 0.1 ng/mL of cytochrome c. When the immobilized antibody is a monoclonal antibody, a monoclonal antibody to be labeled is preferably a clone of a different kind from that of the immobilized antibody. Commercially available antibodies, for example, clone: 6H2.B4, (Becton Dickinson and Company) or clone: 2B5F8 (TECHNE Inc.) may be used.
4) The beads or the cup is preferably washed with buffer containing surfactant such as Tween 20, and then measurement is performed by a method corresponding to the kind of labeling. For example, radioactivity is measured when the antibody is radioactively labeled, or enzymatic activity is measured when the antibody is labeled with an enzyme. Further, when the antibody is labeled with biotin, labeled-avidin is further added and measurement is performed by a method corresponding to the kind of labeling.
5) A calibration curve is created by using the measured value of the sample containing known amounts of cytochrome c, and the amount of cytochrome c in a sample is calculated.

According to the above steps, cytochrome c in the samples is quantified.

Further, the present invention relates to a diagnostic kit for predicting the severity of patients treated with an anti-viral agent such as lamivudine. A kit of the present invention contains a reagent for quantifying cytochrome c, preferably an anti-cytochrome c antibody. For example, the reagent for quantifying cytochrome c by the sandwich method may contain 1) an anti-cytochrome c antibody-coated cup or anti-cytochrome c antibody-coated beads and 2) a labeled anti-cytochrome c antibody, and preferably further contains 3) standard solution of a known concentration of cytochrome c 4) a diluent, and 5) washing solution. Further, if the reagent contains an antibody labeled with an enzyme, the reagent may contain 6) coloring substrate and 7) solution for terminating a reaction.

A method for quantifying cytochrome c and a diagnostic kit disclosed in the present invention can be used in order to examine the severity of hepatitis by quantifying cytochrome c in body fluid of viral hepatitis type B patients treated with an anti-viral agent, preferably lamivudine. The term “severity” used herein includes severity at the time of quantification as well as future severity (prognosis).

A patient with a high value of cytochrome c, specifically, for example, 500 pg/mL or higher, which is quantified by the method and reagent for quantifying cytochrome c disclosed in the present invention, is judged as severe, and a patient with a value of 1,000 pg/mL or higher is judged as severe with higher accuracy. The quantified value of cytochrome c can be used as a guide for choosing more advanced therapies for hepatitis type B, such as administration of interferon, administration of anti-viral agents of different kinds, or administration of steroid drugs.

Cytochrome c is a favorable indicator for examining the severity of patients treated with an anti-viral agent, and can be a useful indicator to find a pathological condition early and accurately.

### EXAMPLES

The present invention will be explained more specifically with reference to the following examples. However, the scope of the present invention is not limited to these examples. “%” refers to “% by mass” unless otherwise indicated.

### Example 1

### Quantification of Cytochrome c by ELISA

Cytochrome c was quantified according to the following procedures.

1) Purification of Anti-Cytochrome c IgG

A rabbit was immunized with rat cytochrome c (Sigma) to obtain antiserum against cytochrome c. Ammonium sulfate was added to the antiserum at a final concentration of 2 M, and the whole was stirred at room temperature (20 to 30° C.) for 5 hours. The stirred solution was centrifuged at 10,000 rpm for 30 minutes and the supernatant was discarded. The precipitate was dissolved by adding 0.1 M phosphate buffer (pH 7.2) and then dialyzed against the same buffer. The dialyzed solution was applied on CNBr-Sepharose 4B (Amersham Pharmacia Biotech) column to which bovine cytochrome c (Sigma) had been bound. The column was washed with 0.01 M Tris-HCl buffer (pH 7.5) containing 0.15 M NaCl, and then anti-cytochrome c IgG was eluted with 0.1 M guanidine hydrochloride. The eluted solution was dialyzed against 0.01 M Tris-HCl buffer (pH 7.5) containing 0.15 M NaCl and thereby, IgG purified product was obtained.

2) Preparation of Anti-Cytochrome c IgG F(ab′)₂

The purified IgG was dialyzed against 0.1 M acetate buffer (pH 4.2). Pepsin (Sigma) was added to the dialyzed IgG solution at the concentration ratio of 1:20 and the whole was allowed to react at 37° C. for 16 hours. The solution to which pepsin was added was adjusted to pH 7.5 by adding 1 N NaOH and then subjected to gel filtration with Sephacryl S-200 (Amersham Pharmacia Biotech) column equilibrated with 0.01 M Tris-HCl buffer (pH 7.5) containing 0.15 M NaCl. A first peak of the fractions obtained from the gel filtration was collected and concentrated and thereby, anti-cytochrome c IgG F(ab′)₂ solution was obtained.

3) Preparation of HRP-Labeled Anti-Cytochrome c Antibody

60 μL of 0.1 M sodium metaperiodate was added to HRP (Toyobo) adjusted to 4 mg/mL and the whole was stirred at room temperature (20to 30° C.) for 20 minutes, and then dialyzed against 0.001 M acetate buffer (pH 4.4). After the dialysis, the dialyzed solution was adjusted to pH 9.0 to 9.5 with 0.2 M sodium carbonate solution. The anti-cytochrome c IgG F(ab′)₂ solution dialyzed against 0.1 M carbonate buffer (pH 9.5) or an anti-cytochrome c monoclonal antibody (clone: 2B5F8, TECHNE) was added to this solution at the concentration of 4 mg/mL, and the whole was stirred at room temperature (20 to 30° C.) for 2hours. Then 50 μL of sodium borohydride solution adjusted to 4 mg/mL was added, and the whole was stirred at 4° C. for 2 hours and then left for stand for 16 hours. This solution was dialyzed against phosphate buffer (pH 7.2) containing 0.15 M NaCl and then subjected to gel filtration by using a Sephacryl S-200. A first peak of the fractions obtained from the gel filtration was collected and diluted with 0.2 M sodium phosphate buffer (pH 5.4) containing 25% by volume of rabbit serum (Nippon Seibutsu Zairyo) and thereby, HRP-labeled anti-cytochrome c antibody was obtained.

4) Preparation of Monoclonal Antibody Against Cytochrome c

110 μg/100 μL of human cytochrome c (Genzyme Techne) was mixed with 55 μL of 2 mg/mL ovalbumin dissolved in 65 mM phosphate buffer (pH 7.5), and 42 μL of 1 mM glutaraldehyde diluted with 65 mM phosphate buffer (pH 7.5) was added thereto. The mixture was stirred at room temperature for 2 hours. Then, the stirred mixture was dialyzed against 0.15 M NaCl at 4° C. for 48 hours and adjuvant were prepared with equal amounts of FCA, and 0.1 mL of these was used to immunize a BALB/C mouse intraperitoneally. In the same manner, immunization was carried out totally three times every two weeks. Two weeks after the third immunization, 50 μg/100 μL of human cytochrome c dissolved in physiological saline was intravenously injected in the tail vein of the mouse. The spleen was removed from the mouse after three days, and according to the conventional method, spleen lymphocytes were subjected to cell fusion with myeloma cells P3X63 Ag8U.1 by the polyethylene glycol method. Screening was carried out by using human cytochrome c as an antigen in order to establish an anti-human cytochrome c monoclonal antibody-producing hybridoma.

The established hybridoma was cultured and proliferated in S-Clone SF-B medium (Sanko Junyaku Co., Ltd.) and then injected into a BALB/C mouse intraperitoneally. Peritoneal fluid was collected after a week. Anti-cytochrome c antibody was purified from the collected peritoneal fluid by using protein A.

5) Preparation of Anti-Cytochrome c Antibody-Immobilized Cup

The anti-cytochrome c monoclonal antibody prepared in 4) or an anti-cytochrome c monoclonal antibody (clone: 6H2.B4, Becton, Dickinson and Company) was adjusted to OD280 nm of 0.1 with 0.01 M Tris-HCl buffer (pH 7.5). 100 μL of the adjusted antibody solution was poured into a polystyrene cup, and was allowed to stand at 4° C. for 16 hours. Then, the cup was washed three times, each for 4 seconds with 0.01 M Tris-HCl buffer (pH 7.5) containing 0.15 M NaCl and 0.01% by volume of Tween20 by using a cup washer. 200 μL of 0.01 M Tris-HCl buffer (pH 7.5) containing 0.5% BSA was added to the washed cup and allowed to stand at 4° C. for 16 hours again and thereby, an antibody-immobilized cup was obtained.

6) Preparation of Standard Antigen

Rat cytochrome c (Sigma) was dissolved in 0.05M Tris-HCl buffer (pH 7.5) containing 2% BSA, 0.01 M EDTA 2Na, 0.1% NaN₃, 0.01% by volume of Tween 20 and 0.15 M NaCl to prepare diluted solution of 50 ng/mL to 50 pg/mL.

7) Measurement

50 μL of 0.05 M Tris-HCl buffer (pH 7.5) containing 2% BSA, 0.01 M EDTA, 0.1% NaN₃, 0.01% by volume of Tween20 and 0.15 M NaCl was poured into the anti-cytochrome c monoclonal antibody-immobilized cup. 50 μL of standard antigens or samples were added to the cup and allowed to react at room temperature (20 to 30° C.) for 1 hour. After the reaction, the cup was washed three times, each for 4 seconds with 0.005 M Tris-HCl buffer (pH 7.5) containing 0.01% by volume of Tween20, 0.15 M NaCl, 0.0015% methyl paraoxybenzoate and 0.005% 2-chloroacetamide by using a cup washer. After the washing, 100 μL of the labeled antibody was added to the cup and allowed to react at room temperature (20 to 30° C.) for 1 hour. After the reaction, the cup was washed three times, each for 4 seconds with 0.005 M Tris-HCl buffer (pH 7.5) containing 0.01% by volume of Tween20, 0.15 M NaCl, 0.0015% methyl paraoxybenzoate and 0.005% 2-chloroacetamide by using a cup washer. After the washing, 100 μL of 0.1 M citrate buffer (pH 4.2) containing 1.5 mg/mL ABTS (2,2-azino-bis-(3-ethylbenzothiazoline-6-sulfonic acid) was added and allowed to react at room temperature (20 to 30° C.) for 1 hour. Then, 100 μL of 0.013% NaN₃ solution was added to terminate the reaction. The absorbance at 405 nm was measured for the color-developed solution by using a spectrophotometer.

8). Calculation of the Amount of Cytochrome c in the Samples

The absorbance value of a blank was subtracted from each of the absorbance values of the standard solutions containing cytochrome c at various standard concentrations and the samples. A standard curve was made by plotting the standard antigen concentration on the horizontal axis and the absorbance of the standard antigen on the vertical axis, respectively. Based on the standard curve, the amount of cytochrome c in the samples was calculated.

### Example 2

### Quantification of Cytochrome c in Sera of Viral Hepatitis Type B Patients Administered with Lamivudine

Cytochrome c levels at the ALT peak in sera of viral hepatitis type B patients who had been administered with lamivudine and developed fulminant hepatitis (11 cases) were determined by using the ELISA system for cytochrome c which was established in Example 1.

As a result, as shown in Table 1, patients whose quantified values of cytochrome c was 1,000 pg/mL or higher, or 500 to 1,000 pg/mL showed severe symptoms subsequently and were obliged to be administered with combination of interferon (IFN), an anti-viral agent of a different kind (Adefovir) and a steroid drug (PSL) or to be administered with interferon repeatedly. On the other hand, patients whose quantified values of cytochrome c were 500 pg/mL or lower had mild symptoms and needed only single administration of IFN or administration of SNMC (Stronger Neo-Minophagen C) or needed no treatment at all.

The above result revealed that the quantified values of cytochrome c in sera of patients could be a good indicator for the subsequent severity of fulminant viral hepatitis type B.

TABLE 1
quantified value of cytochrome c treatment (n = 11)

| | |
|---|---|
| 1000 pg/ml or more | IFN + Adefovir + PSL |
| | IFN + Adefovir |
| | repeated IFN |
| 500-1000 pg/ml | IFN + Adefovir + PSL |
| | repeated IFN |
| 500 pg or less | IFN (3 cases) |
| | SNMC (1 case) |
| | no-treatment (2 case) |

### Example 3

57 cases in which lamivudine was administered for HBV were investigated about usefulness of cytochrome c quantification. This investigation was performed for HBs antigen-positive cases, and all the cases are lamivudine-administered cases. The correlation between hepatitis activity and cytochrome c was analyzed by quantifying at 5 to 6 time points during the course for each case. Lamivudine-stable cases (10 cases) as the control group and mutant-appearing cases (47 cases) as the subject cases were analyzed. Details are listed below.

TABLE 2

| | |
|---|---|
| lamivudine administered cases | 57 cases |
| lamivudine-stable cases | 10 cases |
| mutant-appearing cases | 47 cases |
| HBV-DNA- and liver enzyme-stable cases | 8 cases |
| HBV-DNA-increasing, liver enzyme-stable cases | 8 cases |
| fulminant hepatitis cases | 31 cases |
| no-treatment cases | 5 cases |
| IFN cases | 6 cases |
| adefovir cases | 10 cases |
| entecavir cases | 10 cases |

Each case was analyzed for relationship between a quantified value of cytochrome c at each time point and the subsequent severity.

First, the relationship between the quantified values of cytochrome c before administering lamivudine and the subsequent severity was summarized in Table 3. It was found difficult to predict the appearance of mutants and the subsequent severity from the quantified values of cytochrome c before administering lamivudine.

TABLE 3
Relationship between the quantified values of cytochrome
c before administering lamivudine and the subsequent severity

| | |
|---|---|
| lamivudine-stable cases | 236 (80-894) |
| mutant-appearing cases | |
| HBV-DNA- and liver enzyme-stable cases | 1046 (295-6599) |
| HBV-DNA-increasing, liver enzyme-stable cases | 436 (148-902) |
| fulminant hepatitis cases | |
| no-treatment cases | 101 (78-1024) |
| IFN cases | 255 (95-2049) |
| adefovir cases | 440 (99-1198) |
| Entecavir cases | 292 (80-1304) |

Next, the relationship between the quantified values of cytochrome c when mutants appeared and the subsequent severity was summarized in Table 4. Comparison between the stable cases and the mutant-appearing cases showed that the mutant-appearing cases tended to exhibit high quantified values of cytochrome c, and it was considered that the quantified values of cytochrome c probably represented increase in cell death in the liver caused by the appearance of mutants.

Further, in the mutant-appearing cases, the group that needed treatment of some kind tended to show high quantified values of cytochrome c, compared with the cases that did not lead to fulminant hepatitis or the group that developed fulminant hepatitis but was able to be followed up without treatment. Therefore, it was demonstrated that the quantified values of cytochrome c are useful for prediction of the subsequent severity.

TABLE 4
Relationship between the quantified values of cytochrome
c when mutants appeared and the subsequent severity

| | |
|---|---|
| lamivudine-stable cases | 100 (71-170) |
| mutant-appearing cases | |
| HBV-DNA- and liver enzyme-stable cases | 184 (88-3170) |
| HBV-DNA-increasing, liver enzyme-stable | 163 (74-296) |
| cases fulminant hepatitis cases | |
| no-treatment cases | 229 (83-369) |
| IFN cases | 507 (86-1847) |
| adefovir cases | 514 (57-1499) |
| entecavir cases | 171 (80-1529) |

Further, by the investigation on the cases that led to fulminant hepatitis, an apparent significant difference was found between the group that was able to be followed up without treatment and the group that needed treatment of some kind (Table 5). Therefore, it was considered that the quantified values of cytochrome c were useful to decide strategy of treatment.

TABLE 5
Quantified values of cytochrome c during the exacerbation
period and response to the treatment

| | |
|---|---|
| no-treatment cases | 253 (111-3210) |
| IFN cases | 1024 (99-1473) |
| adefovir cases | 1316 (148-9859) |
| entecavir cases | 900 (232-2048) |

In the cases where the values of cytochrome c are high, early administration of interferon or other anti-viral agent leads to lifesaving treatment. Therefore, the values of cytochrome c can be an indicator for the treatment course of getting severe in the case that the patients subjected to lamivudine-administration led to fulminant hepatitis.

While the invention has been described in detail with reference to preferred embodiments thereof, it will be apparent to one skilled in the art that various changes can be made, and equivalents employed, without departing from the scope of the invention. Each of the aforementioned documents, including the foreign priority document, JP 2003-383408, is incorporated by reference herein in its entirety.

1. A method for predicting severity of viral hepatitis type B in patients treated with an anti-viral agent, wherein cytochrome c in body fluid of the viral hepatitis type B patients is quantified.

2. The method according to claim 1, wherein the anti-viral agent is lamivudine.

3. The method according to claim 1 or 2 comprising the steps of:
(a) quantifying cytochrome c in body fluid of the viral hepatitis type B patients treated with an anti-viral agent; and
(b) determining that the severity is high when the quantified value of cytochrome c is high.

4. The method according to claim 1 or 2, wherein cytochrome c is quantified by an immunochemical method.

5. A diagnostic kit for predicting severity of viral hepatitis type B in patients treated with an anti-viral agent, comprising a reagent for quantifying cytochrome c.

6. The kit according to claim 5, wherein the anti-viral agent is lamivudine.

7. The kit according to claim 5 or 6, wherein the reagent for quantifying cytochrome c is an anti-cytochrome c antibody.
